SEQUENCE LISTING (22 sequences)

SEQ ID NO: 1 - DNA, 26 bp, artificial sequence
  primer
agtcccaaaggcaaacaacccacttc

SEQ ID NO: 2 - DNA, 27 bp, artificial sequence
  primer
tgctggaggctgaggtatttctgtctc

SEQ ID NO: 3 - DNA, 25 bp, artificial sequence
  primer
cttgctgcagaagtgggtggaggaa

SEQ ID NO: 4 - DNA, 21 bp, artificial sequence
  primer
ctgcagtgtgggtttcgggca

SEQ ID NO: 5 - DNA, 18 bp, artificial sequence
  primer
cccccggcggcaatagca

SEQ ID NO: 6 - DNA, 20 bp, artificial sequence
  primer
tcggcgccggggagatacat

SEQ ID NO: 7 - DNA, 26 bp, artificial sequence
  primer
gcgtcctgggaagggagatccggagc

SEQ ID NO: 8 - DNA, 26 bp, artificial sequence
  primer
ttgaggggcatcgtcgcgggaggctg

SEQ ID NO: 9 - DNA, 20 bp, artificial sequence
  primer
tagtcagctgaagtgggaca

SEQ ID NO: 10 - DNA, 20 bp, artificial sequence
  primer
acgacaggtgcactttgtag

SEQ ID NO: 11 - DNA, 20 bp, artificial sequence
  primer
ctacaggggcacttaaccca

SEQ ID NO: 12 - DNA, 20 bp, artificial sequence
  primer
agagctgaatcgctcagagc

SEQ ID NO: 13 - DNA, 20 bp, artificial sequence
  primer
tgcctgagaaagagaaccag

SEQ ID NO: 14 - DNA, 20 bp, artificial sequence
  primer
atggcaggatgaacaaacac

SEQ ID NO: 15 - DNA, 22 bp, artificial sequence
  primer
gtcagctcaatgccgacctccg

SEQ ID NO: 16 - DNA, 21 bp, artificial sequence
  primer
gcagtggcgtcctggtactgc

SEQ ID NO: 17 - DNA, 19 bp, artificial sequence
  primer
ggcagcagcaagcattcct

SEQ ID NO: 18 - DNA, 18 bp, artificial sequence
  primer
gcccaacacccccagtca

SEQ ID NO: 19 - DNA, 21 bp, artificial sequence
  primer
agcctcatgaagagccttcca

SEQ ID NO: 20 - DNA, 19 bp, artificial sequence
  primer
tccggaagaaacccttgca

SEQ ID NO: 21 - DNA, 18 bp, artificial sequence
  primer
aggaggaggcagagcaca

SEQ ID NO: 22 - DNA, 20 bp, artificial sequence
  primer
ctggtatggcacaggtgatg

(19) United States
(12) Patent Application Publication
(10) Pub. No.: US 2012/0270314 A1
(43) Pub. Date: Oct. 25, 2012

(54) NON-TUMORIGENIC EXPANSION OF PLURIPOTENT STEM CELLS

(75) Inventors: Dah-Ching Ding, Hualien (TW); Tang-Yuan Chu, Hualien (TW)

(73) Assignee: BUDDHIST TZU CHI GENERAL HOSPITAL, Hualien (TW)

(21) Appl. No.: 13/433,681

(22) Filed: Mar. 29, 2012

Related U.S. Application Data
(60) Provisional application No. 61/476,838, filed on Apr. 19, 2011.

(51) Int. Cl. C12N 5/0735; C12N 5/0775; C12N 5/071

(52) U.S. Cl. 435/366

(57) ABSTRACT

A method for expansion of human embryonic stem (hES) cells in a medium including human umbilical cord-derived mesenchymal stem cells (HUCMSCs) as a feeder is provided. The human embryonic stem cells (hES) maintain the features of embryonic stem cells in the medium, such as pluripotency, unlimited undifferentiated proliferation and normal karyotypes. Also provided is a method for non-tumorigenic expansion of the human embryonic stem cells (hES) that is free from forming teratoma.

### BACKGROUND OF THE INVENTION

1. Field of the Invention

The present invention relates to expansion of human pluripotent stem cells, and particularly relates to non-tumorigenic expansion of human embryonic stem (hES) cells.

2. Description of Related Art

Human embryonic stem (hES) cells are pluripotent, and they have great ability to differentiate into almost all cell types of the adult. These cells therefore hold great promise for regenerative medicine. The special properties that make these hES cells desirable include immortality, pluripotency, and unlimited undifferentiated growth. Pluripotent embryonic stem cells are traditionally cultured on a layer of feeder cells such as mouse embryonic fibroblasts (MEFs) to keep them in an undifferentiated state. This feeder layer acts essentially to support the cells; and hES cell cultures are commonly cultured on a layer of the feeder layer until differentiation is desired. Unfortunately, these mouse feeder support cells are often associated with contamination to the hES cell cultures without much functional impact on them, thus rendering the hES cells that have been cultured on mouse feeder cells unsuitable for use clinically. It has been reported that human pluripotent stem (hPS) cells cultured without feeders soon die, or differentiate into a heterogeneous population of committed cells.

Numerous reports exist demonstrating that there have been attempts to replace the feeder or support cells using cell-free components, or at least to avoid non-human components or cells. The replacements have not shown long-term promising results, and such attempts have proven insufficient to support robust, continued propagation of cells.

Furthermore, it has been shown that, in feeder-cell-free cultures, hES cells grown in medium replacements do form differentiated cells around the hES colonies, which is an indication that optimal conditions have not been achieved.

Therefore, a need exists for an alternative strategy for culturing hES cells by using another source of feeder cells that does not cause contamination and form teratomas.

### SUMMARY OF THE INVENTION

In one embodiment, a method for expansion of human pluripotent stem cells is provided by co-culturing the human pluripotent stem cells with umbilical cord-derived stem cells. The umbilical cord-derived stem cells form a feeder layer in a medium for the expansion of the human pluripotent stem cells, and maintain the human pluripotent stem cells in an undifferentiated state.

In one embodiment, the expansion of the human pluripotent stem cells is non-tumorigenic expansion, such that the human pluripotent stem cells are free from forming teratoma.

In another embodiment, the umbilical cord-derived stem cells are positive for CD10, CD13, CD29, CD44, CD73, CD90, CD166 or HLA-ABC, but negative for CD1q, CD3, CD34, CD45, CD56, CD117 or HLA-DR. Also, the umbilical cord-derived stem cells have osteogenic or adipogenic differentiability.

In another embodiment, the umbilical cord-derived stem cells are human umbilical cord-derived mesenchymal stem cells (HUCMSCs), and are derived from Wharton's jelly of a human umbilical cord.

In another embodiment, the human pluripotent stem cells are positive for alkaline phosphatase (AP), Oct-4, SSEA-1, SSEA-4, TRA-1-60, TRA-1-81, NANOG, SOX2, NF-200, brachyury, ATBF1 or MAP2, and express GDF9, GATA4, HAND1 or TUJ-1 genes. Also, MYC is down-regulated in the human pluripotent stem cells.

In another embodiment, the human pluripotent stem cells are human embryonic stem (hES) cells, and form embryiod bodies.

In another embodiment, a medium for expansion of human pluripotent stem cells is provided, wherein the medium includes umbilical cord-derived stem cells that form a feeder layer in the medium. In one embodiment, the umbilical cord-derived stem cells in the medium are human umbilical cord-derived mesenchymal stem cells (HUCMSCs).

In another embodiment, a kit for expansion of human embryonic stem (hES) cells is provided, wherein the kit comprises a medium including umbilical cord-derived stem cells, and instructions for the use thereof. In one embodiment, the umbilical cord-derived stem cells in the medium of the kit are human umbilical cord-derived mesenchymal stem cells (HUCMSCs).

### BRIEF DESCRIPTION OF THE DRAWINGS

FIG. 1 shows morphology, immunotyping and in vitro differentiation of HUCMSC. Cells of Wharton's jelly (WJ) growing from explants are fibroblast-like with a spindle-shaped morphology (FIG. 1A). Flow cytometry of rapidly dividing HUCMSCs showed negative for CD1q, CD3, CD34, CD45, CD56, CD117 and HLA-DR, and positive for CD10, CD13, CD29, CD44, CD73, CD90, CD166 and HLA-ABC (FIG. 1B). Upon adipogenic differentiation, the cells formed neutral lipid vacuoles and contained numerous Oil-Red-O positive lipid droplets (FIG. 1C, the upper panel). In osteogenic medium, the cells broadened to form a mineralized matrix, which was strongly stained with Alizarin Red S (FIG. 1C, the lower panel) after three to four weeks of cultivation. Expression of genes specific for adipogenic (PPARγ) and osteogenic (osteopontin) differentiation was showed by RT-PCR analysis with GAPDH as a positive control (FIG. 1D). Scale bars represent 1000 μm in the left panel of FIG. 1A and 100 μm in the right two panels of FIG. 1A and in FIG. 1C.

FIG. 2 shows morphology of undifferentiated human ES cell colonies grown on HUCMSC and MEF feeders. Pictures of the hES colony grown on HUCMSC (FIG. 2A) and MEF (FIG. 2B) feeder layers are shown. A magnification of colony grown on HUCMSC revealed typical hES cell morphology with high nucleus:cytoplasm ratio (FIGS. 2C and 2D). Scale bars represent 1000 μm in FIGS. 2A and 2B and 100 μm in FIGS. 2C and 2D.

FIG. 3 shows phenotypes of human ES cells cultured on HUCMSC. Immunostaining of the hES colony with specific antibodies revealed strong expression of alkaline phosphatase (FIG. 3A), Oct4 (FIG. 3B), SSEA-4 (FIG. 3C), TRA-1-60 (FIG. 3D), and TRA-1-81 (FIG. 3E). A representative normal karyotype (46, XX) was observed in ES cells after 20 passages of continuous culture on HUCMSC (FIG. 3F). Scale bars represent 1000 μm.

FIG. 4 shows fluorescence immunostaining of embryonic bodies (EB) derived from hES. Embryonic bodies are shown under phase contrast microscope (FIG. 4A), and by immunostaining with antibodies against NF-200 (FIG. 4B), brachyury (FIG. 4C), ATBF1 (FIG. 4D), and MAP2 (FIG. 4E). Scale bars represent 1000 μm.

FIG. 5 shows expression of differentiation markers in hES cells on different feeders. FIG. 5A illustrates expression of genes that are specific for germ cell (GDF9), endoderm (GATA4), mesoderm (HAND)) and ectoderm (TUJ-1) by means of RT-PCR. GAPDH was used as a control. FIG. 5B illustrates semi-quantitative analysis of gene expression shown in FIG. 5A.

FIG. 6 shows teratomas developed from hES cells with a change of the feeder cells from HUCMSC to MEF. FIG. 6A shows teratoma (indicated by the arrow) readily developed from hES cells after change of the feeder from HUCMSC to MEF on NOD-SCID mice. Histological section revealed ribbon of melanocytes with a retina-like structure (FIG. 6B), neurotube-like structures (FIGS. 6C-6E), odontogenic epithelium (FIG. 6F), neuroepithelium (FIG. 6G), immature cartilage (FIG. 6H) and immature squamoid epithelium (FIG. 6I). Scale bars represent 100 μm.

FIG. 7 shows expression of pluripotent genes in hES cells cultured on HUCMSC and MEF. FIG. 7A illustrates RT-PCR analysis of OCT4, NANOG SOX2 and MYC in hES cells (ES) cultured on HUCMSC (WJ) and MEF with GAPDH as the internal control. FIG. 7B illustrates semi-quantitative analysis of gene expression shown in FIG. 7A. FIG. 7C illustrates the C-MYC protein measured by Western blot analysis. FIG. 7D illustrates the mRNA levels of MYC measured by qRT-PCR as revealed by folds of the internal control. EB stands for the embryoid body.

### DETAILED DESCRIPTION OF THE PREFERRED EMBODIMENTS

Various specific details are provided herein to provide a more thorough understanding of the invention.

### RT-PCR and Quantitative RT-PCR of the Gene and Other Differentiation Marker Genes

Undifferentiated or differentiated hES cells that had been cultured on HUCMSCs or MEF feeder layers were removed mechanically and treated with RLT lysis buffer (Qiagen). The first strand of cDNA was synthesized using a SuperScript III One-Step RT-PCR kit (Invitrogen) following the manufacturer's instructions. Table 1 presents the sequence, annealing temperature and product size of each pair of primers. All PCR samples were analyzed by electrophoresis on 2% agarose gel that contained 0.5 μg/ml ethidium bromide (Sigma). For quantitative RT-PCR (qRT-PCR) analysis, FastStart universal SYBR green master (ROX, Roche, USA) gene expression assays was used in an ABI Step One Plus system (Applied Biosystems), with GAPDH used as an internal control. The sequences of primers and annealing temperatures are shown in Table 1.

TABLE 1

| | | | Annealing | Product |
|---|---|---|---|---|
| Gene | Sense (5′-3′) | Antisense (5′-3′) | Temp (° C.) | size (bp) |
| Nanog | AGTCCCAAAGGCAAACAACCCACTTC | TGCTGGAGGCTGAGGTATTTCTGTCTC | 55 | 164 |
| Oct3/4 | CTTGCTGCAGAAGTGGGTGGAGGAA | CTGCAGTGTGGGTTTCGGGCA | 55 | 171 |
| SOX2 | CCCCCGGCGGCAATAGCA | TCGGCGCCGGGGAGATACAT | 55 | 448 |
| MYC | GCGTCCTGGGAAGGGAGATCCGGAGC | TTGAGGGGCATCGTCGCGGGAGGCTG | 55 | 328 |
| GDF9 | TAGTCAGCTGAAGTGGGACA | ACGACAGGTGCACTTTGTAG | 55 | 278 |
| GATA4 | CTACAGGGGCACTTAACCCA | AGAGCTGAATCGCTCAGAGC | 60 | 157 |
| Hand1 | TGCCTGAGAAAGAGAACCAG | ATGGCAGGATGAACAAACAC | 55 | 273 |
| Tuj-1 | GTCAGCTCAATGCCGACCTCCG | GCAGTGGCGTCCTGGTACTGC | 55 | 559 |
| GAPDH | GGCAGCAGCAAGCATTCCT | GCCCAACACCCCCAGTCA | 60 | 136 |
| PPAR-r | AGCCTCATGAAGAGCCTTCCA | TCCGGAAGAAACCCTTGCA | 60 | 120 |
| osteopontin | AGGAGGAGGCAGAGCACA | CTGGTATGGCACAGGTGATG | 60 | 150 |

### Isolation and Expansion of HUCMSCs

Human umbilical cord samples (20 cm in length, 20 g in weight) were collected in sterile boxes that contained Hanks' balanced salt solution (HBSS; Gibco/BRL 14185-052). The protocols for collecting and using human umbilical cord were approved by the Institutional Review Board of Tzu-Chi University Hospital. Written informed consent was obtained from pregnant women before labor pain.

Collected human umbilical cord tissues were washed three times in Ca²⁺ and Mg²⁺-free phosphate buffered saline (Dulbecco's PBS, Life Technology). They were mechanically cut along the midline, and the umbilical arteries, vein and outlining amniotic membrane were dissociated from the WJ. The jelly was then cut into pieces that were smaller than 0.5 cm³, treated with trypsin/EDTA (Sigma, St. Louis, USA), and incubated for 30 min at 37° C. in a 95% air/5% CO₂ humidified atmosphere. The explants were then cultured in Dulbecco's Modified Eagle Medium (DMEM) that contained 10% human umbilical cord blood serum (CBS) and antibiotics and left undisturbed for five to seven days to allow the cells to migrate from the explants.

Characteristics of HUCMSCs Derived from Human Umbilical Cord

The MSC specific surface markers were characterized by flow cytometric analysis. The cells were detached using 2 mM EDTA in phosphate buffered saline (PBS), washed and incubated with the appropriate antibody that was conjugated with fluorescein isothiocyanate (FITC) or phycoerythrin (PE). The antibodies were CD1q, CD3, CD10, CD13, CD14, CD29, CD31, CD34, CD44, CD45, CD49b, CD49d, CD56, CD73, CD90, CD105, CD117, CD166, HLA-ABC and HLA-DR (BD, PharMingen). Then, the cells were analyzed using a Becton Dickinson flow cytometer (Vantage SE, Becton Dickinson, San Jose, Calif.).

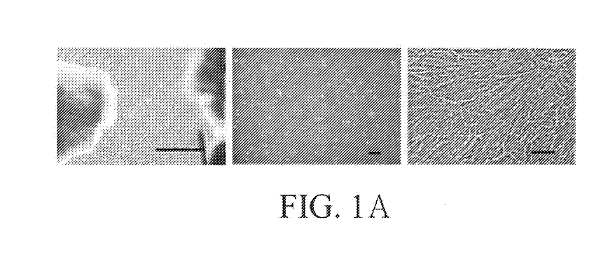

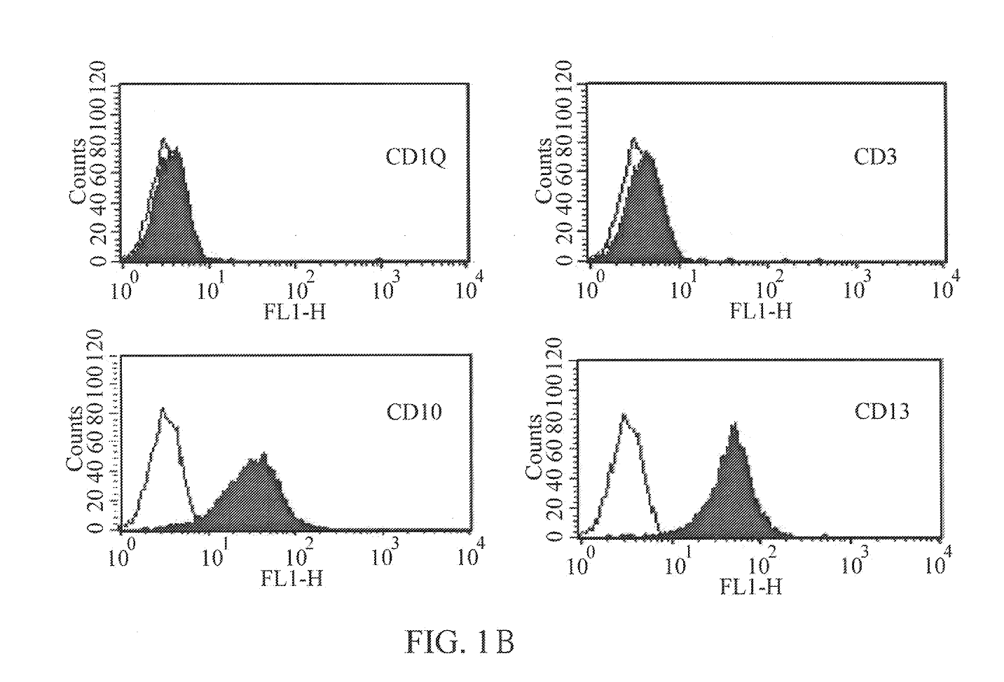

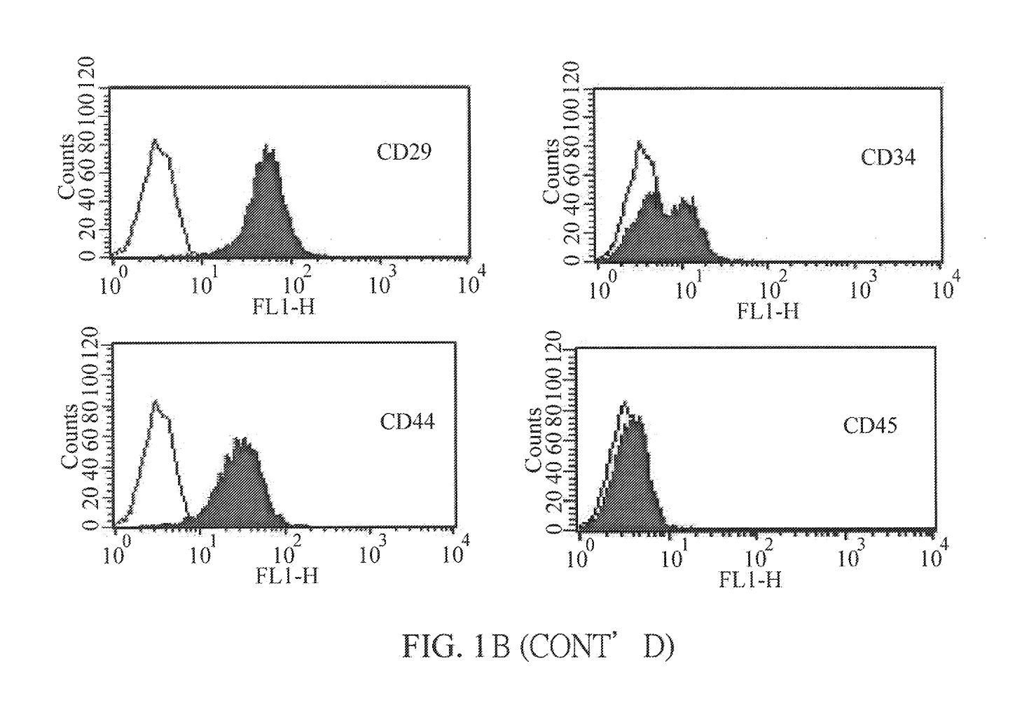

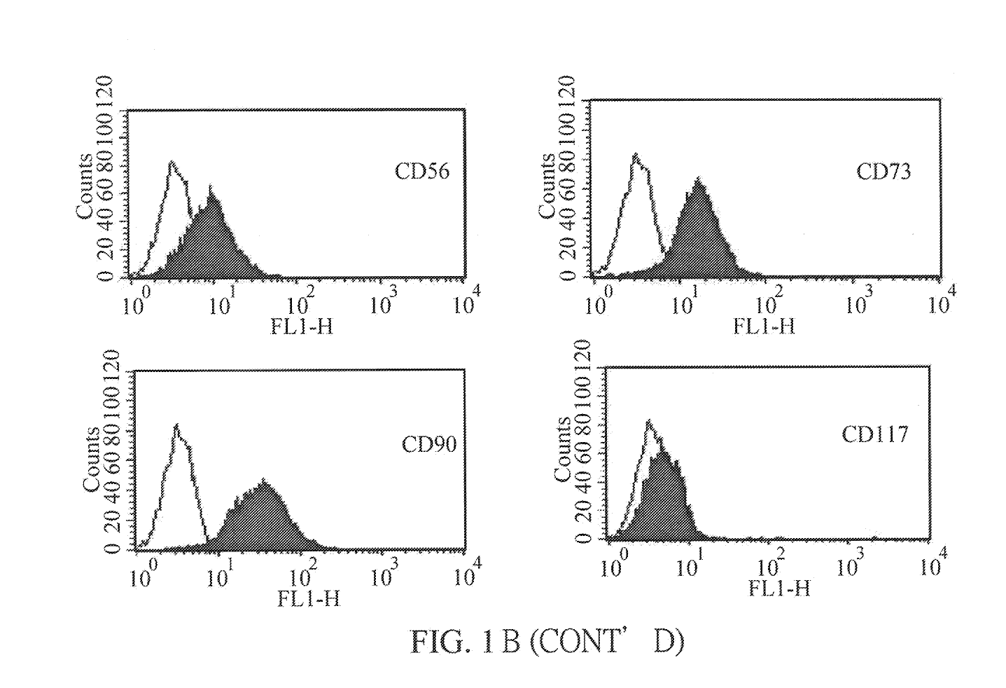

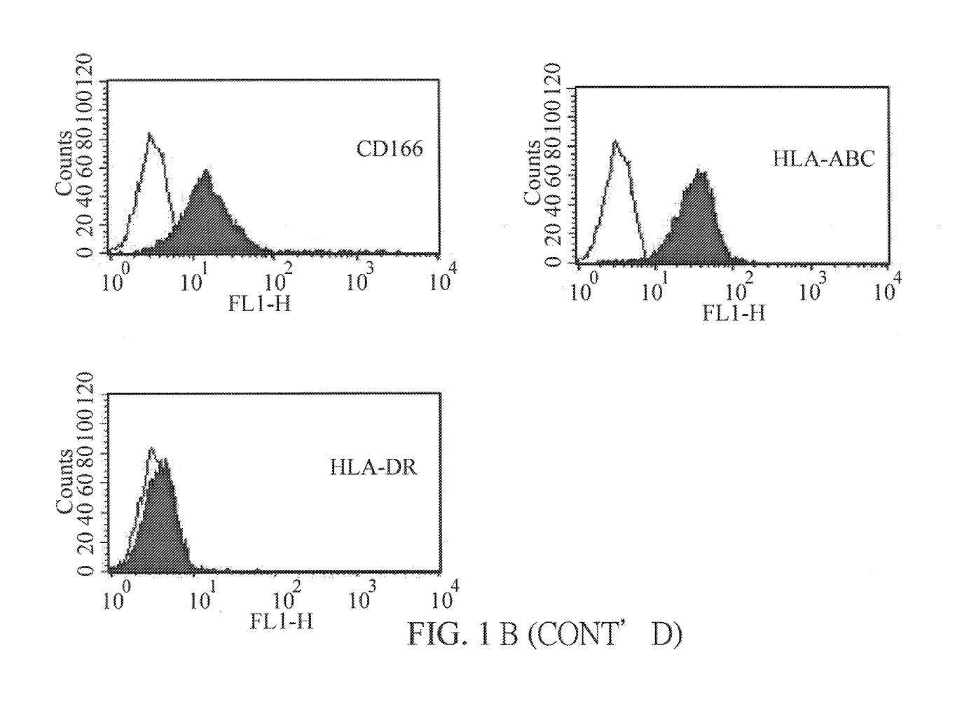

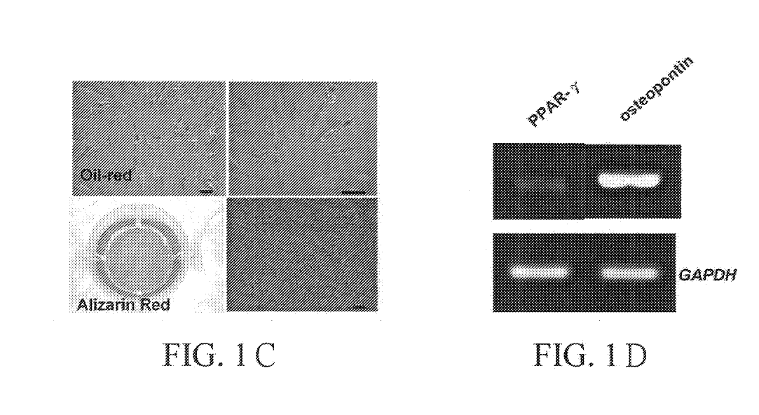

Upon the first culturing of WJ tissue pieces, attached growing cells with a spindle-shaped morphology migrated from the explants (FIG. 1A). These cells divided rapidly with a doubling time of 28 h, and underwent more than 25 passages (equivalent to over 40 population doublings), without spontaneous differentiation. They were negative for CD1q, CD3, CD34, CD45, CD56, CD117, and HLA-DR, and positive for CD10, CD13, CD29, CD44, CD73, CD90, CD166 and HLA-ABC (FIG. 1B). These observations demonstrate that cells that are isolated from WJ of the human umbilical cord have the same surface markers as mesenchymal stem cells (MSCs).

### In Vitro Differentiation of HUCMSCs to Osteocytes and Adipocytes

To induce osteogenic and adipogenic differentiations, HUCMSCs were transferred to an osteogenic medium (DMEM supplemented with 10% CBS, 0.1 μmol/L dexamethasone, 10 mmol/L β-glycerol phosphate, 50 μmol/L ascorbate) and adipogenic medium (DMEM supplemented with 10% CBS, 1 μmol/L dexamethasone, 5 μg/mL insulin, 0.5 mmol/L isobutylmethylxanthine and 60 μmol/L indomethacin) for three weeks. The potential for osteogenesis was evaluated by determining the mineralization of calcium by staining with Alizarin Red S (Sigma, USA). To assess adipogenic differentiation, intracellular lipid droplets were observed under a microscope and the lipid droplet was verified by staining with Oil Red O.

Adipogenic differentiation of HUCMSC was apparent two weeks after incubation with an adipogenic medium supplement. At the end of the second week, changes were evident in the cell morphology, and in the formation of neutral lipid vacuoles: almost all cells contained many Oil Red-O-positive lipid droplets (FIG. 1C). Similarly, the induction of differentiation in the osteogenic medium caused the treated cells to grow rapidly and contain mineralized matrices, which were strongly stained by Alizarin Red-S, indicating deposition of calcium after three to four weeks of cultivation (FIG. 1C). Expression of adipogenic (PPARγ) and osteogenic (Osteopontin) genes was evident in RT-PCR analysis (FIG. 1D).

### Characterization of HUCMSC-Co-Cultured hES Cell Lines

The TW1 cell line (P22, i.e., the twenty-second passage) was obtained from Biomedical Technology and Device Research Laboratories, Industrial Technology Research Institute, and initially cultured on MEFs, as directed by the supplier, i.e., the Food Industry Research and Development Institute, FIRDI, Taiwan. Either MEFs (P3, the third passage) or HUCMSCs after mitomycin-C deactivation were used as feeder cells for culturing the hES cells. They were plated at a density of 200,000 cells per 9.4 cm² per well in six-well plates. The hES cell culture medium comprised 80% (v/v) knockout (KO) DMEM, 20% (v/v) KO serum replacement, 2 mM L-glutamine, 10 mM nonessential amino acids (all from Invitrogen), 50 μM B-mercaptoethanol (Sigma), and 4 ng/mL bFGF.

The hES cells were characterized by immunocytochemistry using fluorescence-labeled antibodies specific for undifferentiated hES cells, which were SSEA-4, SSEA-1, TRA-1-60, TRA-1-81 and Oct-4 (ES Cell Characterization Kit; Chemicon). Initially, hES cells were cultured on chamber slides (Nunc, Denmark) or sterile cover glasses (Assistent, Germany) in culture dishes with feeder cells. At specified times on days three to seven following passage, the colonies of the hES cells were subjected to immunofluorescence staining. Briefly, cells were fixed with 4% paraformaldehyde and then underwent several procedures, including permeabilization (0.1% Triton X-100), blocking (4% normal goat serum), treatment with primary antibody (1:10-1:50 dilution), three washings, treatment with fluorescent-labeled secondary antibody, three more washings, covering with a coverslip and mounting. Alkaline phosphatase (AP) staining was conducted using the ES Cell Characterization Kit (Chemicon).

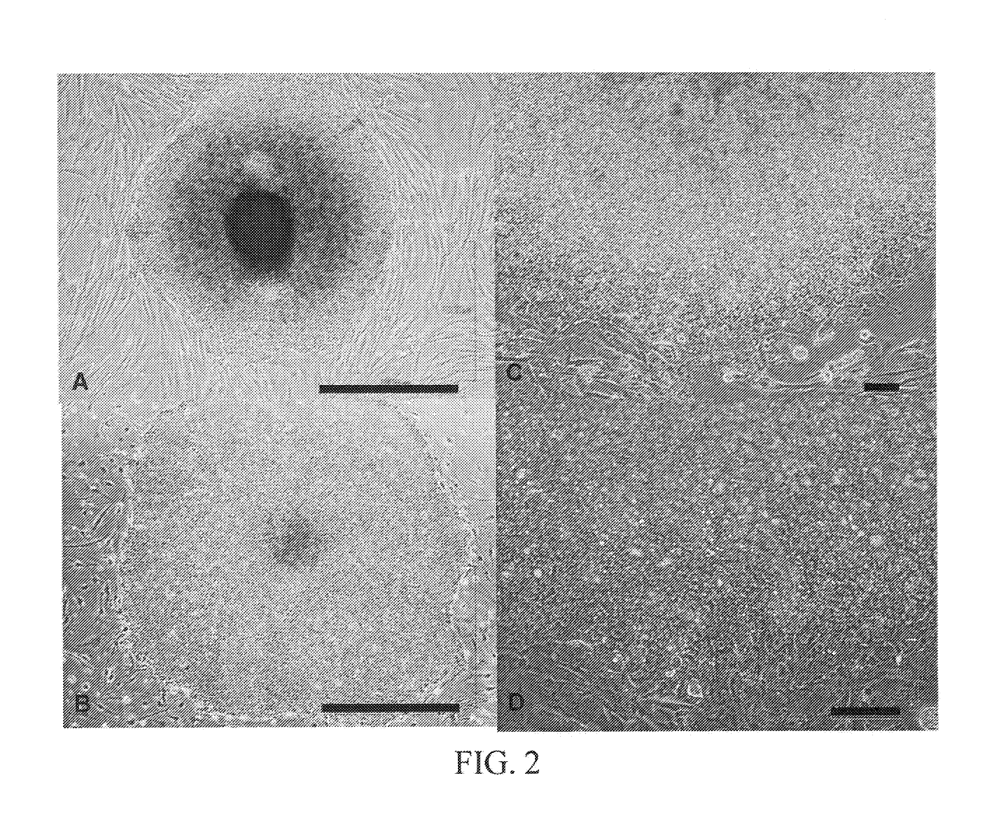

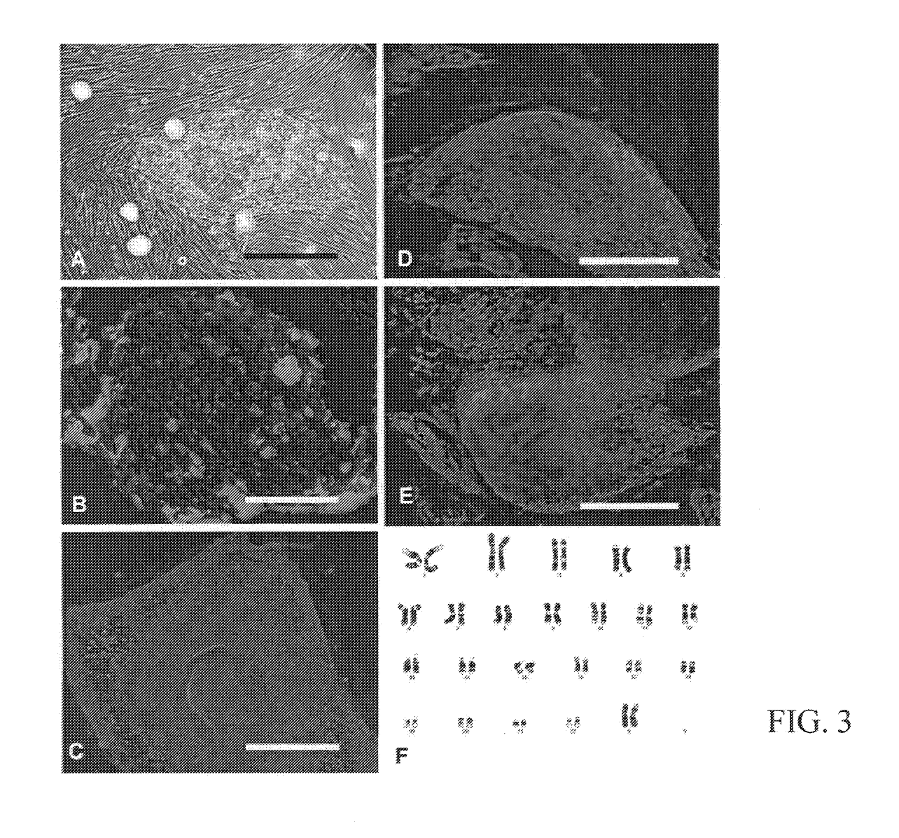

The hES cells that were transferred to the HUCMSCs feeders formed colonies effectively and continued to proliferate with a doubling time of 36 hours. The morphology of colonies differed slightly from that of those that were cultured on MEF (FIGS. 2A and 2B). However, the individual human ES cell morphology cultured on HUCMSC remained the same as on MEF. The cells remained round and small, with a high nucleus:cytoplasm ratio, and the notable presence of one to three nucleoli (FIGS. 2C and 2D). The TW1 hES cells on HUCMSCs feeders expressed AP, Oct-4, SSEA-4, TRA-1-60 and TRA-1-81 markers (FIGS. 3A-3E).

The karyotypes of cells were studied at passage 40. On day seven after passage, hES cells were treated with 0.1 μg/ml colcemid (Gibco) for 4 h. After the cells were washed, they were treated with either 0.25% trypsin for 3-5 min or collagenase type IV for 8 min, pipetted and harvested. They were then fixed and mounted on glass slides. The metaphases were analyzed using the standard G-banding method in a certified cytogenetic laboratory. At passage 40, the TW1 hES cells on HUCMSCs feeders revealed normal karyotypes of 46, XX (FIG. 3F).

### In Vitro Pluripotency of HUCMSC-Co-Cultured hES Cell Line

Before differentiation, hES cells were collected and resuspended in the hES cell medium in the absence of bFGF. The hES cells were then cultured in low attachment six-well plates as aggregates in suspension. After five days, fetal bovine serum (FBS) (5% final) was added. Aggregated hES cells usually formed an embryoid body (EB) after seven to ten days, and mature (cystic) EBs subsequently emerged from 20% to 80% of the formed EBs. The resultant solid or cystic EB was then plated on gelatin-treated chamber slides or 35 mm culture dishes for further differentiation and was then subsequently treated similarly to those cells that were grown directly in an adherent culture. After fixation, the differentiated hES cells were studied by immunocytochemistry using fluorescence-labeled antibodies specific for three embryonic germ layers, which were MAP2, NF200 (Chemicon) for ectoderm, brachyury for mesoderm and ATBF1 (Santa Cruz) for endoderm.

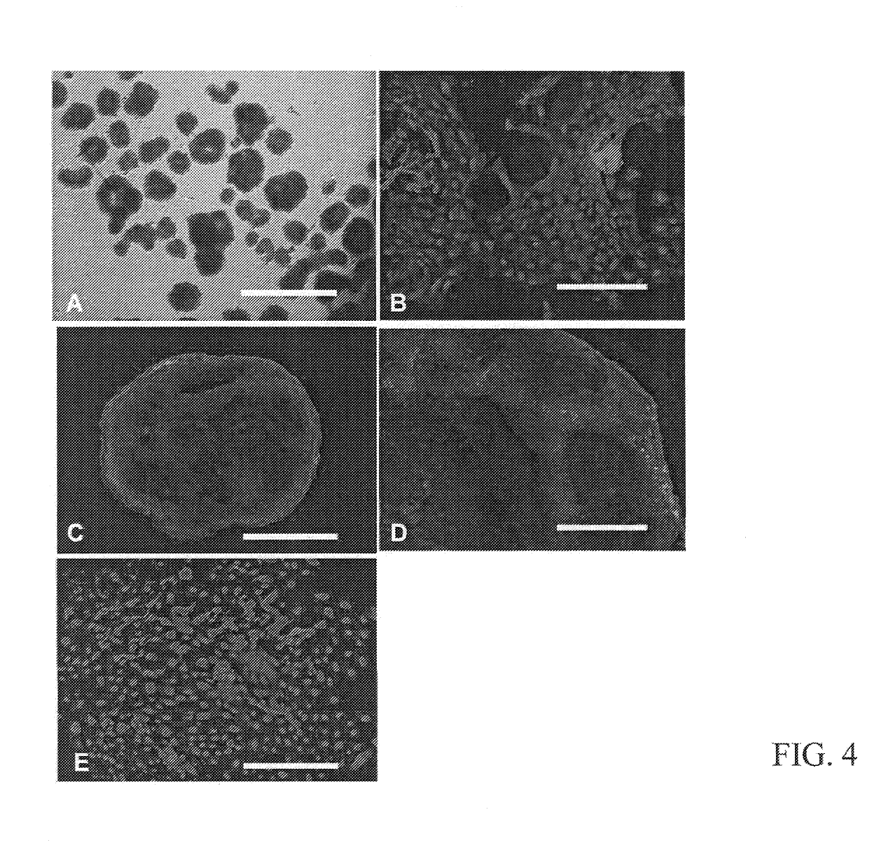

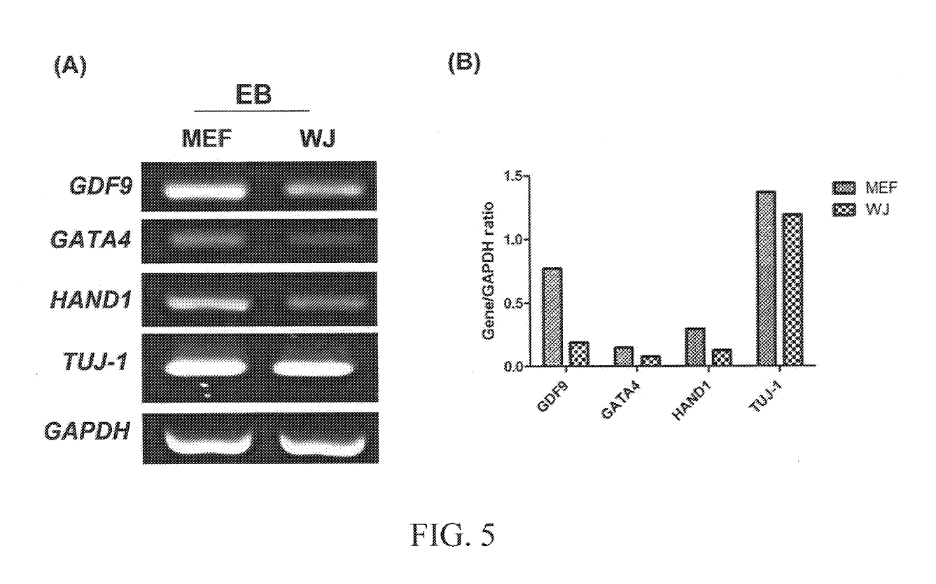

Like those grown on MEFs, hES cells cultured with HUCMSC feeders formed EBs when cultured in suspension. Within these EBs, cell differentiation that represented three embryonic germ layers was observed. These cells expressed ectodermal markers (MAP-2 and NF-200), mesodermal marker (brachyury) and endodermal marker (ATBF1), as indicated by immunohistochemistry (FIG. 4). Cells that were harvested from seven to ten day-old EBs expressed such genes as GDF9 (germ cell related), GATA4 (endoderm), Hand1 (mesoderm) and Tuj-1 (ectoderm), as indicated by RT-PCR (FIG. 5).

### Non-Tumorigenesis and In Vivo Pluripotency of HUCMSC-Co-Cultured hES Cell Line

hES cells were detached with mechanical slicing using glass capillaries, pelleted, resuspended in PBS with Matrigel (BD Biosciences) (1:1) and injected into the back subcutaneous tissue (n=17) or renal capsule (n=4) of non-obese diabetic-severe combined immunodeficiency (NOD-SCID) mice. Cells were counted using a hemocytometer and suspended in PBS with Matrigel (1:1) in various concentrations. hES cells were kept on ice less than 45 min for optimal viability prior to injection. The teratoma formation was followed up by palpation and resulting tumors were dissected, fixed, embedded in paraffin and processed for histology.

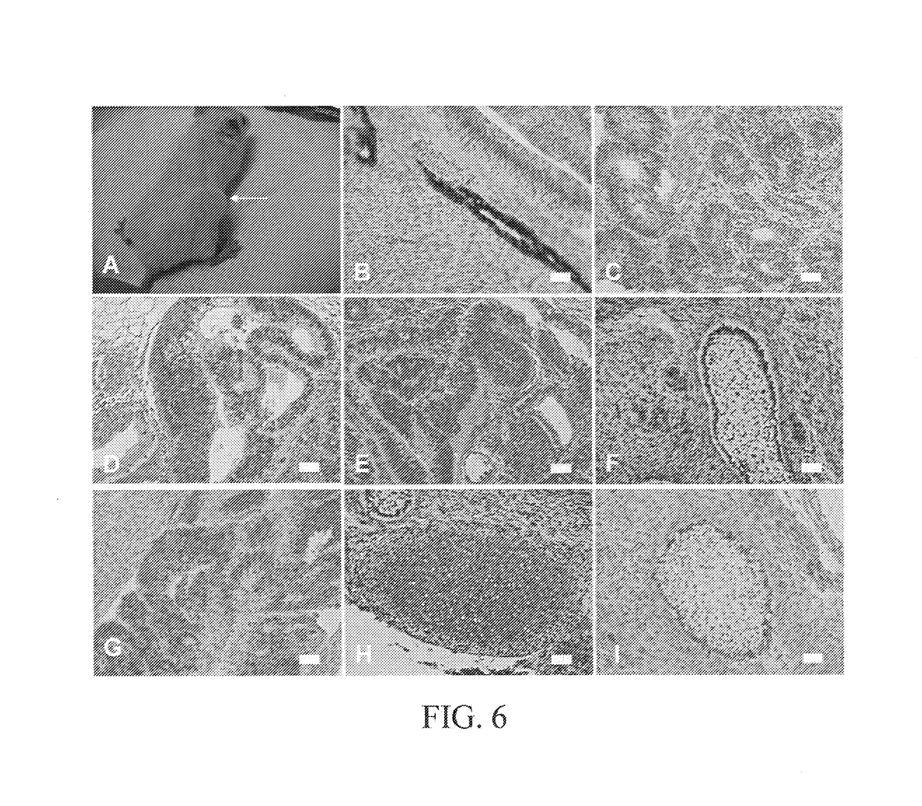

The developmental potential of cells after long-term culture on HUCMSC feeders was investigated in vivo using a xenograft model. In extensive trials with 21 different transplantations in both NOD-SCID (n=18) and nude (n=3) mice, no teratoma growth was observed during long-term followed up for over three months (Tables 2 and 3). However, teratomas were clearly observed upon transient (for days) subsequent culturing on MEF feeders. The histologies of the teratomas that were derived from the hES cells before and after they were transferred to the HUCMSC feeder did not differ (Table 2 and FIG. 6).

TABLE 2
Summary of xenograft experiments with TW1 human embryonic
stem cells alternatively cultured on MEF and HUCMSC as feeders

| Xenograft | Feeder | Teratoma/injection |
|---|---|---|
| date | layer | numbers |
| January 2008 | MEF | 2/2 |
| April 2008-June 2009 | HUCMSC | 0/21 |
| March 2009 | MEF | 2/2 |

TABLE 3
Trials of xenograft tumorigenesis of human embryonic stem cells
co-cultured with HUCMSCs

| | Injected cell | | Mouse | Site of |
|---|---|---|---|---|
| Xenograft date | numbers | Mouse | number | injection |
| Apr. 18, 2008 | 2 × 10⁵ | NOD-SCID | 2 | s.c.* |
| Jun. 9, 2008 | 3 × 10⁵ | NOD-SCID | 2 | s.c. |
| Jul. 8, 2008 | 5 × 10⁵ | NOD-SCID | 2 | s.c. |
| Jul. 16, 2008 | 2 × 10⁶ | NOD-SCID | 2 | s.c. |
| Jan. 8, 2009 | 1.5 × 10⁶ | nude mice | 2 | s.c. |
| Jan. 22, 2009 | 3 × 10⁵ | nude mice | 1 | s.c. |
| Mar. 6, 2009 | 1 × 10⁶ | NOD-SCID | 2 | s.c |
| Mar. 19, 2009 | 5 × 10⁶ | NOD-SCID | 3 | s.c. |
| Apr. 30, 2009 | 3 × 10⁷ | NOD-SCID | 2 | s.c. |
| Jun. 19, 2009 | 7 × 10⁵ | NOD-SCID | 4 | renal capsule |

*s.c.: subcutaneous

Down-Regulation of MYC in hES Co-Cultured with HUCMSC

Cells were lysed in the lysis buffer (150 mM NaCl, 50 mM Tris-HCl [pH 7.4], 1% Nonidet P-40) plus proteinase inhibitor cocktail (Roche, Indianapolis, Ind.). Proteins were electrophoresed on 10% sodium dodecyl sulfate-polyacrylamide gel electrophoresis (SDS-PAGE) and then transferred to a nitrocellulose membrane (Hybond-C Super; Amersham, Little Chalfont, UK). The membranes were incubated with anti-c-myc (2 μg/m) or anti-α-actin (1:10,000; Sigma-Aldrich) monoclonal antibodies. HRP-conjugated goat anti-mouse IgG (Jackson Immuno-Research Laboratories) was used as the secondary antibody. Bound antibodies were detected using enhanced chemiluminescence reagents (ECL; Amersham).

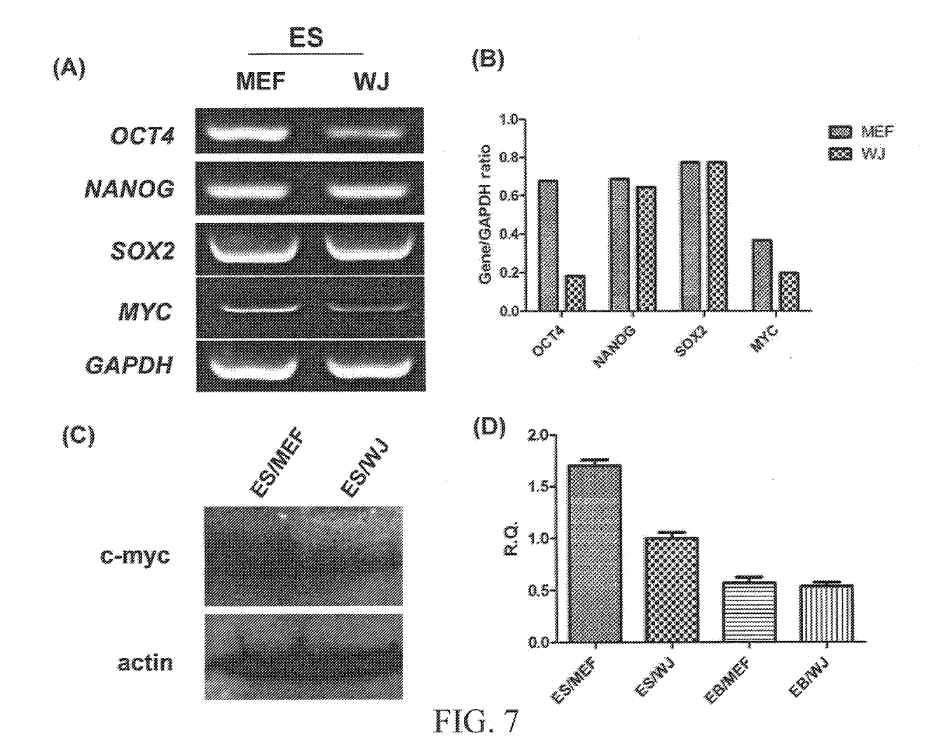

The expression of key pluripotency genes in hES on the two different feeders was further tested. Markers of undifferentiated stem cells, such as Oct-4, Nanog, and Sox2 were readily expressed as indicated by RT-PCR (FIGS. 7A and 7B). Lower expressions of the homeobox gene OCT4 and proto-oncogene MYC were observed in hES co-cultured with HUCMSC (FIGS. 7A and 7B). This down-regulation of MYC was further proved by Western blot analyses and quantitative RT-PCR (FIGS. 7C and 7D).

What is claimed is:

1. A method for expansion of human pluripotent stem cells, comprising co-culturing the human pluripotent stem cells with umbilical cord-derived stem cells.

2. The method of claim 1, wherein the umbilical cord-derived stem cells form a feeder layer in a medium for the expansion of the human pluripotent stem cells.

3. The method of claim 1, wherein the umbilical cord-derived stem cells are human umbilical cord-derived mesenchymal stem cells (HUCMSCs).

4. The method of claim 3, wherein the human umbilical cord-derived mesenchymal stem cells (HUCMSCs) are derived from Wharton's jelly of a human umbilical cord.

5. The method of claim 1, wherein the umbilical cord-derived stem cells maintain the human pluripotent stem cells in an undifferentiated state.

6. The method of claim 1, wherein the umbilical cord-derived stem cells are positive for one or more of CD10, CD13, CD29, CD44, CD73, CD90, CD166 and HLA-ABC.

7. The method of claim 1, wherein the umbilical cord-derived stem cells are negative for one or more of CD1q, CD3, CD34, CD45, CD56, CD117 and HLA-DR.

8. The method of claim 1, wherein the umbilical cord-derived stem cells have osteogenic or adipogenic differentiability.

9. The method of claim 1, wherein the human pluripotent stem cells are human embryonic stem (hES) cells.

10. The method of claim 1, wherein the human pluripotent stem cells are positive for one or more of alkaline phosphatase (AP), Oct-4, SSEA-1, SSEA-4, TRA-1-60, TRA-1-81, NANOG, SOX2, and a differentiation marker, and wherein the differentiation marker is one or more of NF-200, brachyury, ATBF1 and MAP2.

11. The method of claim 1, wherein the human pluripotent stem cells express one or more of differentiation genes, and wherein the differentiation genes are GDF9, GATA4, HAND1 or TUJ-1 genes.

12. The method of claim 1, wherein MYC is down-regulated in the human pluripotent stem cells.

13. The method of claim 1, wherein the expansion of the human pluripotent stem cells is non-tumorigenic expansion.

14. The method of claim 13, wherein the human pluripotent stem cells are free from forming teratoma.

15. The method of claim 1, wherein the human pluripotent stem cells form embryiod bodies.

16. A medium for expansion of human pluripotent stem cells, comprising umbilical cord-derived stem cells.

17. The medium of claim 16, wherein the umbilical cord-derived stem cells form a feeder layer in the medium.

18. The medium of claim 16, wherein the umbilical cord-derived stem cells are human umbilical cord-derived mesenchymal stem cells (HUCMSCs).

19. A kit for expansion of human embryonic stem (hES) cells, comprising a medium including umbilical cord-derived stem cells, and instructions for the use thereof.

20. The kit of claim 1, wherein the umbilical cord-derived stem cells are human umbilical cord-derived mesenchymal stem cells (HUCMSCs).
